(19) United States
(12) Patent Application Publication
(10) Pub. No.: US 2023/0228657 A1
(43) Pub. Date: Jul. 20, 2023

(54) METHOD FOR ANALYZING DYNAMIC CHARACTERISTICS OF CARBON COMPOSITE MATERIALS WITH RESPECT TO THE CARBON FIBER ANGLE

(71) Applicant: PUKYONG NATIONAL UNIVERSITY INDUSTRY-UNIVERSITY COOPERATION FOUNDATION, Busan (KR)

(72) Inventor: Chan-Jung Kim, Busan (KR)

(21) Appl. No.: 17/715,767

(22) Filed: Apr. 7, 2022

(30) Foreign Application Priority Data
Jan. 14, 2022 (KR) 10-2022-0005865

(51) Int. Cl. G01N 3/02; G01N 33/00; G06F 30/20

(52) U.S. Cl. CPC G01N 3/02; G01N 33/00; G06F 30/20; G01N 2033/0003; G06F 2111/10

(57) ABSTRACT

This application relates to a method for analyzing the dynamic characteristics of carbon composite materials. In the method, a specimen for a specific carbon fiber orientation is prepared and a modal test is performed on the specimen to obtain data and analyze the characteristics of the specimen on the basis of the data. To solve conventional carbon composite material dynamic characteristic analysis methods for carbon composite materials, the proposed method can determine the orientation of carbon fiber orientation exhibiting desired dynamic characteristics by predicting various system parameters at the state of designing, i.e., before the manufacture of a carbon composite material. Especially, the method predicts system parameters such as structural stiffness and viscous damping coefficient which are very sensitive to the orientation of carbon fiber, using only data of a single reference orientation, and reflects the prediction results on the design of a carbon composite material.

### CROSS REFERENCE TO RELATED APPLICATION

The present application claims priority to Korean Patent Application No. 10-2022-0005865, filed Jan. 14, 2022, the entire content of which is incorporated herein for all purposes by this reference.

### BACKGROUND

### Technical Field

The present disclosure relates to a method for analyzing the dynamic characteristics of carbon composite materials. In conventional dynamic behavior analysis of carbon composite materials, carbon fiber specimens having a specific orientation are prepared and a characteristic test such as a modal test is performed on the specimens to obtain data of each specimen and analyze the characteristics on the basis of the data. The present disclosure is to solve problems with such a conventional dynamic behavior analysis method for carbon composite material. The conventional method has limitations in that the method is neither able to predict various system parameters before the manufacture of carbon composite materials nor determine the orientation of carbon fiber having the desired dynamic behavior at the designing stage. To solve this problem, the present disclosure proposes an orientation-dependent dynamic behavior analysis method for carbon fiber composite materials, the method being capable of accurately predicting system parameters of carbon composite materials for various carbon fiber orientations in a simple manner while using only limited information such as system parameters only for a specific carbon fiber orientation.

In addition, as described above, in order to overcome such limitations of the conventional carbon composite material dynamic characteristic analysis methods, such as it was not possible to predict various system parameters in the design stage of carbon composite materials to determine the carbon fiber orientations having desired dynamic characteristics in advance, the present disclosure is configured to predict the structural stiffness and viscous damping coefficient values for various carbon fiber orientations, using only data of a carbon fiber reference orientation. That is, the present disclosure relates to a carbon composite material dynamic behavior analysis method capable of predicting system parameters of carbon composite materials for various carbon fiber orientations by using data of only a single orientation of carbon fiber and reflecting the parameters on the design of carbon composite materials.

### Description of Related Technology

In general, in order to understand the dynamic characteristics of a composite structure or to select a configuration of laminated carbon in a carbon-based composite (CBC) structure, it is important to accurately understand various system parameters.

In addition, system parameters for the dynamic characteristics of the CBC structure may be represented as modal parameters in the frequency domain, where all modal parameters vary depending on the carbon fiber orientations.

Accordingly, various methods for analyzing dynamic characteristics of carbon composite materials using frequency analysis and modal parameters have been proposed in the related art.

Here, as an example of the related art for an apparatus and method for analyzing the dynamic characteristics of a carbon composite material using frequency analysis and modal parameters as described above, first, for example, is “Sensitivity analysis apparatus using a frequency response and a sensitivity analysis method using the same” as shown in Korean Patent Publication No. 10-2223538.

More specifically, the above-mentioned Korean Patent Publication No. 10-2223538 discloses including an exciter for setting an excitation pattern by control, and applying a physical force to one side of a test object according to the set excitation pattern; a first sensor contacting one side of the test object and measuring a physical force applied to the test object by the exciter; a second sensor contacting the other side of the test object and collecting vibrations of the test object caused by a physical force; setting the excitation pattern by controlling the exciter, and converting the physical force signal measured by the first sensor and the vibration signal collected by the second sensor according to the set excitation pattern into a frequency domain signal to calculate the frequency response function of the test object, and a sensitivity analysis apparatus for calculating a sensitivity index to a physical external variable of a test object based on a frequency response function, and the test object consists of an object formed by including materials arranged to have unidirectionality at a specific angle. Disclosed is a sensitivity analysis apparatus using frequency response configured to more accurately analyze the physical characteristics of an object by analyzing it in consideration of the directionality of the internal structure.

In addition, as another example of the related art for an apparatus and method for analyzing the dynamic characteristics of a carbon composite material as described above, for example, “Modal damping coefficient measurement apparatus and method for measuring modal damping coefficient using the same” as presented in Korean Patent Publication No. 10-2051746.

More specifically, the aforementioned Korean Patent No. 10-2051746 relates to a modal damping coefficient measuring apparatus and a modal damping coefficient measuring method using the same. The patent above-mentioned discloses including a test object for calculating a modal damping coefficient; an exciter setting an excitation pattern by control and applying a physical force to one side of the test object according to the set excitation pattern; a sensor being in contact with the other side of the test object and collecting a vibration signal generated from the test object by a physical force; and modal damping that converts the physical force signal applied by the exciter and the vibration signal collected by the sensor into a frequency domain signal to calculate a frequency response function extracts a resonance point based on the frequency response function, and calculates a modal damping coefficient for the resonance point. Disclosed are a modal damping coefficient measuring device and modal damping coefficient using the modal damping coefficient measuring device configured to reduce the difference from the real situation affected by various external input patterns and accurately analyze the physical characteristics of the test object by measuring the modal damping coefficient through various input excitation patterns.

### SUMMARY

The present disclosure relates to a method for analyzing the dynamic characteristics of carbon composite materials. In general, conventional methods for analyzing dynamic characteristics of carbon composite materials are mostly configured to prepare a specimen for a specific carbon fiber orientation and perform a characteristic test such as a modal test to obtain data and analyze same. The present disclosure is to solve problems of methods for analyzing dynamic characteristics of carbon composite materials according to the related art, which have limitations and have yet to be presented. The present disclosure relates to a method of determining a carbon fiber orientation having desired dynamic characteristics by predicting various system parameters in advance in a design step of a carbon composite material before manufacturing a product. More specifically, the present disclosure relates to the method for analyzing dynamic characteristics of carbon composite material according to carbon fiber angle configured to accurately predict system parameters of carbon composite material with various angles with a relatively simple configuration using only data in a single carbon fiber orientation.

In addition, as described above, before making a product, predicting various system variables in the design stage of the carbon composite material in advance to determine the carbon fiber orientations having desired dynamic characteristics was not presented. In order to solve such limitations of the dynamic characteristic analysis methods of composite materials, the present disclosure is configured to predict the structural stiffness and viscous damping coefficient values for the carbon fiber orientations at different angles using only the carbon fiber orientation data for reference direction, for a structural stiffness and viscous damping coefficient, which are system parameters that are very sensitive to the carbon fibers orientations, among the dynamic characteristics of carbon composite materials. Accordingly, the present disclosure relates to a method of analyzing the dynamic characteristics of carbon composite materials according to the carbon fiber angle, which is configured to predict system parameters of carbon composite materials for various carbon fiber orientations and reflect them in the design of carbon composite materials by using only data in a single carbon fiber orientation.

In order to achieve the above objectives, according to the present disclosure, the method for analyzing the dynamic characteristics of the carbon composite material according to the carbon fiber angle includes: a data collection step in which data on various measurement values and system parameters are collected for the carbon composite material to be analyzed is performed; a system parameter calculation step in which a process of calculating a viscous damping coefficient and a stiffness coefficient for a carbon angle of the carbon composite material to be analyzed is respectively performed based on the data collected in the data collection step; a sensitivity calculation step in which a process of calculating the sensitivity of the viscosity damping coefficient and the structural stiffness coefficient is respectively performed based on the viscosity damping coefficient and the structural stiffness coefficient calculated in the system parameter calculation step; a system parameter approximation step in which a process of approximating changes in the viscous damping coefficient and the structural stiffness coefficient according to a change in the carbon fiber angle using a curve-fitting method is performed based on the sensitivity calculated in the sensitivity calculation step; and a system parameter estimation step in which processing of estimating a system parameter for an arbitrary carbon fiber orientation is performed based on the processing result of the system parameter approximation step, and the method is configured to be performed through a computer or dedicated hardware.

Here, the data collection step is configured to perform a modal test to collect various predetermined system parameters or to receive data collected in advance through the modal test after preparing a specimen having a preset reference carbon fiber angle (θ₁) with respect to the carbon composite material to be analyzed.

In addition, the system parameter calculation step is configured to perform a process of calculating a normalized equivalent viscosity damping coefficient (C̅_{eq,i}) and an equivalent stiffness coefficient (k̅_{eq,i}) in the ith mode, respectively, using the following equation.

$${\overline{c}}_{eq , i} ( {θ}_{j} ) = {( \frac{1}{2 {\tilde{ς}}_{i , C} ( {θ}_{j} ) {ω}_{{n}_{i} , C} ( {θ}_{j} )} + \frac{1}{2 {\tilde{ς}}_{i , M} ( {θ}_{j} ) {ω}_{{n}_{i} , M} ( {θ}_{j} )} )}^{- 1}$$

$${\overline{k}}_{eq , i} ( {θ}_{j} ) = {( {ω}_{{n}_{i} , C} ( {θ}_{j} ) )}^{2} + {( {ω}_{{n}_{i} , M} ( {θ}_{j} ) )}^{2}$$

(Where, θⱼ is the carbon fiber angle, ξ_{i,C} and ξ_{i,M} are the modal damping ratios of the carbon fiber and polymer matrix in the ith mode, respectively, and ω_{ni,C} and ω_{ni,M} are the resonance frequency of carbon fiber and polymer matrix in the ith mode, respectively) In addition, the sensitivity calculation step is configured to perform a process of calculating the sensitivity of the viscous damping coefficient and the structural stiffness coefficient at a specific carbon fiber angle θⱼ with respect to the reference carbon fiber angle θ₁, respectively, using the following equation.

$$\frac{{\overline{c}}_{C , i} ( {θ}_{j} )}{{\overline{c}}_{C , i} ( {θ}_{1} )} \approx \frac{{\overline{c}}_{eq , i} ( {θ}_{j} )}{{\overline{c}}_{eq , i} ( {θ}_{1} )}$$

$$\frac{{\overline{k}}_{C , i} ( {θ}_{j} )}{{\overline{k}}_{C , i} ( {θ}_{1} )} = \frac{{\overline{k}}_{eq , i} ( {θ}_{j} )}{{\overline{k}}_{eq , i} ( {θ}_{1} )}$$

(Where, C̅_{Ci}(θ₁) and C̅_{C,i}(θⱼ) are the viscous damping coefficients of the carbon fiber at θ₁ and θⱼ, respectively, C̅_{eq,i}(θ₁) and C̅_{eq,i}(θⱼ) are the equivalent viscous damping coefficients of the carbon fiber at θ₁ and θⱼ, respectively, k̅_{C,i}(θ₁) and k̅_{C,i}(θⱼ) are the stiffness modulus of the carbon fiber at θ₁ and θⱼ, respectively, C̅_{eq,i}(θ₁) and C̅_{eq,i}(θⱼ) are the equivalent stiffness modulus of carbon fiber at θ₁ and θⱼ, respectively)

Furthermore, the system parameter approximation step is configured to perform the process of approximating each and rearranging each system parameter using the following equation, in which the increase or decrease of the viscous damping coefficient and the structural stiffness coefficient at the jth increased carbon fiber angle ϕⱼ from the reference carbon fiber angle ϕ₁ using a curve-fitting function (Γ_{c̅,i}, Γ_{k̅,i}).

$${Γ}_{\overline{c} , i} ( {ϕ}_{j} ) \approx \frac{{\overline{c}}_{C , i} ( {ϕ}_{j} )}{{\overline{c}}_{C , i} ( {ϕ}_{1} )}$$

$${Γ}_{\overline{k} , i} ( {ϕ}_{j} ) \approx \frac{{\overline{k}}_{C , i} ( {ϕ}_{j} )}{{\overline{k}}_{C , i} ( {ϕ}_{1} )}$$

In addition, the system parameter estimation step is configured to perform processing of estimating system parameters for an arbitrary carbon fiber orientation using the following equation.

In addition, according to the present disclosure, provided is a computer-readable recording medium on which a computer program configured to execute a method for analyzing dynamic characteristics of a carbon composite material according to the carbon fiber angle described above is recorded.

Furthermore, according to the present disclosure, the system for analyzing the dynamic characteristics of a carbon composite material includes: an input unit for receiving various data including information about the modal test result and each system parameter of the carbon composite material to be analyzed; an analysis unit performing a process of estimating a system parameter according to a change in the carbon fiber angle of the carbon composite material to be analyzed based on the data input through the input unit; an output unit for outputting data input through the input unit and a processing result of the analysis unit; and a control unit for controlling the overall operation of the analysis system. Provided is a dynamic characteristic analysis system of a carbon composite material according to the angle of carbon fiber, in which the analysis unit is configured to perform estimating system parameters according to changes in the carbon fiber angle of the carbon composite material to be analyzed using the dynamic characteristics analysis method of the carbon composite material according to the above-described carbon fiber angle.

Here, the analysis system is configured by installing and configuring a program dedicated to an information processing device, including a PC or a laptop computer, so that it can be implemented without the need to construct separate hardware.

As described above, according to the present disclosure, with respect to the structural stiffness and viscous damping coefficient, which are system parameters very sensitive to the carbon fiber orientation among the dynamic characteristics of the carbon composite material, values of structural stiffness and viscosity damping coefficient for carbon fiber orientation of other angles can be predicted, respectively, using only the data of the carbon fiber orientation for the reference angle. Accordingly, provided is a method for analyzing dynamic characteristics of a carbon composite material according to an angle of a carbon fiber configured to predict system parameters of carbon composite materials for various carbon fiber orientations and reflect them in the design of carbon composite materials using only single carbon fiber orientation data. Therefore, before making a product, various system variables may be predicted in advance in the design step of the carbon composite material to easily determine the direction of the carbon fiber having desired dynamic characteristics.

In addition, according to the present disclosure, provided is a method for analyzing dynamic characteristics of a carbon composite material according to an angle of a carbon fiber configured to accurately predict system parameters of a carbon composite material having various angles in a relatively simple configuration using only data in a single carbon fiber orientation, as described above. A method for determining the direction of carbon fiber having desired dynamic characteristics by predicting various system variables in advance in the design stage of carbon composite material before making a product, in which most of the process is to produce specimens for specific carbon fiber orientations and perform characteristic tests to obtain and analyze data, has not been presented and can solve the limitations of the methods for analyzing the dynamic characteristics of carbon composite materials of the related art.

### BRIEF DESCRIPTION OF THE DRAWINGS

FIG. 1 is a flowchart schematically showing the overall configuration of a method for analyzing the dynamic characteristics of a carbon composite material according to an angle of carbon fiber according to an embodiment of the present disclosure.

FIG. 2 is a view schematically showing the overall configuration of the carbon composite material specimen produced for the verification test of the method for analyzing the dynamic characteristics of the carbon composite material according to the carbon fiber angle according to an embodiment of the present disclosure.

FIG. 3 is a diagram schematically showing a position of a sensor attached to a specimen.

FIG. 4 is a view showing the mode parameters measured for each of the specimens in five carbon fiber orientations in a table.

FIGS. 5A and 5B are graphs showing a change in structural stiffness according to the orientation of carbon fiber, respectively.

FIGS. 6A and 6B are graphs showing the change in the viscous damping coefficient according to the direction of the carbon fiber, respectively.

FIG. 7 is a view showing the increase or decrease of the structural stiffness and the viscous damping coefficient by using the carbon fiber angle variable ϕⱼ in a table.

FIGS. 8A and 8B are graphs showing changes in structural stiffness and viscous damping coefficient using the carbon fiber angle variable ϕⱼ.

FIG. 9 is a table showing representative curve-fitting functions for each system variable obtained using a curve-fitting function.

FIGS. 10A-10D are views showing the results of comparing the prediction result of the stiffness coefficient change according to the carbon fiber angle predicted using the representative curve-fitting function shown in FIG. 9 with the actual measured value.

FIGS. 11A-11D are views showing the results of comparing the prediction result of the change in the viscous damping coefficient according to the carbon fiber angle predicted using the representative curve-fitting function shown in FIG. 9 with the actual measured value.

FIG. 12 is a view showing the results of calculating a relative error with respect to the actual measurement data in order to compare the accuracy of the actual measurement data and the predicted data in a table.

FIG. 13 is a block diagram schematically showing the overall configuration of the carbon composite material specimen produced for the verification test of the method for analyzing the dynamic characteristics of the carbon composite material according to the carbon fiber angle according to an embodiment of the present disclosure.

### DETAILED DESCRIPTION

Various devices and methods have been proposed to analyze the dynamic characteristics of carbon composite materials using frequency analysis and modal parameters, but the contents of the related art as described above have the following limitations.

That is, in the design stage of a carbon composite material, if a method for predicting various system variables before making a product is presented, it is expected that the carbon fiber orientation of a carbon composite material having desired dynamic characteristics can be effectively determined.

However, the dynamic characteristic analysis apparatus and methods of the related art as described above, are configured to prepare a specimen for a specific carbon fiber orientation to be known, perform a characteristic test such as a modal test, obtain all necessary data such as system parameters, and analyze the data obtained through the characteristic test. Accordingly, in order to obtain a carbon composite material having desired dynamic characteristics, a plurality of specimens should be manufactured while changing the direction of carbon fiber, and characteristic tests and analyses should be repeated, thereby increasing time and cost.

Therefore, in order to solve the limitations of the methods for analyzing the dynamic characteristics of carbon composite materials of the related art as described above, for example, it is desirable to present a method for analyzing the dynamic characteristics of a carbon composite material with a new composition that can accurately predict the dynamic characteristics of a carbon composite material with a relatively simple composition using the limited information, such as system parameter information at a specific carbon fiber angle, but no device or method that satisfies all such needs is yet presented.

Hereinafter, with reference to the accompanying drawings, a specific embodiment of the method for analyzing the dynamic characteristics of the carbon composite material according to the angle of the carbon fiber according to the present disclosure will be described.

Here, it should be noted that the content described below is only one embodiment for carrying out the present disclosure, and the present disclosure is not limited to the content of the embodiment described below.

In addition, it should be noted that in the following description of an embodiment of the present disclosure, parts identical to or similar to those of the related art or determined to be easily understood and implemented at the level of those skilled in the art are omitted.

The present disclosure relates to a method for analyzing the dynamic characteristics of carbon composite materials. In general, conventional methods for analyzing dynamic characteristics of carbon composite materials are mostly configured to prepare a specimen for a specific carbon fiber orientation and perform a characteristic test such as a modal test to obtain data and analyze same. The present disclosure is to solve problems of methods for analyzing dynamic characteristics of carbon composite materials according to the related art, which has limitations and has not yet been proposed. The present disclosure relates to a method of determining a carbon fiber orientation having desired dynamic characteristics by predicting various system parameters in advance in a design step of a carbon composite material before manufacturing a product. More specifically, the present disclosure relates to a method for analyzing dynamic characteristics of carbon composite material according to carbon fiber angle configured to accurately predict system parameters of carbon composite material with various angles with a relatively simple configuration using only data in a single carbon fiber orientation.

In addition, as described above, prior to making a product, predicting various system variables in the design stage of the carbon composite material in advance to determine the carbon fiber orientations having desired dynamic characteristics was not presented. In order to solve such limitations of the dynamic characteristic analysis methods of composite materials, the present disclosure is configured to predict the structural stiffness and viscous damping coefficient values for the carbon fiber orientations at different angles using only the carbon fiber orientation data for reference direction, for structural stiffness and viscous damping coefficient, which are system parameters that are very sensitive to the carbon fibers orientations, among the dynamic characteristics of carbon composite materials. Accordingly, the present disclosure relates to a method of analyzing the dynamic characteristics of carbon composite materials according to the carbon fiber angle, which is configured to predict system parameters of carbon composite materials for various carbon fiber orientations and reflect them in the design of carbon composite materials by using only data in a single carbon fiber orientation.

Hereinafter, with reference to the accompanying drawings, a specific embodiment of the method for analyzing the dynamic characteristics of the carbon composite material according to the angle of the carbon fiber according to the present disclosure will be described.

First, in general, a carbon composite material is composed of carbon fiber and a polymer matrix, and as such, the one-degree-of-freedom (DOF) structure may be represented by the following [Equation 1].

m\ddot{x}(t)+c_{eq}x̅(t)+k_{eq}x(t)=0[Equation 1]

Here, in the above [Equation 1], m is a mass, and c_{eg} and k_{eg} are an equivalent damping coefficient and an equivalent spring coefficient, respectively.

The above two equivalent system parameters can be expressed by the linear combination of the two main components of the carbon composite material structure, that is, carbon fiber and a polymer matrix, as formulated in the following [Equation 2] and [Equation 3] and can be expressed together.

$${c}_{eq} = {( \frac{1}{{c}_{C}} + \frac{1}{{c}_{M}} )}^{- 1} \qquad [ Equation 2 ]$$

$${k}_{eq} = {k}_{C} + {k}_{M} \qquad [ Equation 3 ]$$

Here, in [Equation 2] and [Equation 3], c_{C} and c_{M} are the damping coefficients of the carbon fiber and the polymer matrix, respectively, and kc and km are the spring coefficients of the carbon fiber and the polymer matrix, respectively.

In addition, the above [Equation 1] can be transformed into modal coordinates by normalizing the system parameters (c_{eg}, k_{eg}) with m, and the normalized system parameters can be expressed using modal parameters.

That is, the normalized system parameters are shown in [Equation 4] and [Equation 5] below, and the transformed governing equations are expressed as [Equation 6] below.

$$\frac{{c}_{eq}}{m} = {( \frac{1}{2 {\tilde{ς}}_{C} {ω}_{n , C} )} + \frac{1}{2 {\tilde{ς}}_{M} {ω}_{n , M}} )}^{- 1} \qquad [ Equation 4 ]$$

$$\frac{{k}_{eq}}{m} - {( {ω}_{n , C} )}^{2} + {( {ω}_{n , M} )}^{2} \qquad [ Equation 5 ]$$

$$\ddot{x} ( t ) + {( \frac{1}{2 {\tilde{ς}}_{C} {ω}_{n , C}} + \frac{1}{2 {\tilde{ς}}_{M} {ω}_{n , M}} )}^{- 1} \dot{x} ( t ) + ( {( {ω}_{n , C} )}^{2} + {( {ω}_{n , M} )}^{2} ) x ( t ) = 0 \qquad [ Equation 6 ]$$

Here, in the above [Equation 4] to [Equation 6], ξ_{C} and ξ_{M} are the modal damping ratios of the carbon fiber and polymer matrix, respectively, and ω_{n,C} and ω_{n,M} are the resonance frequencies of the carbon fibers and polymer matrix, respectively.

In addition, the carbon composite material should be extended to a multi-DOF system in practice because the dynamic characteristics of the carbon composite material can be representative of the multi-DOF model instead of the 1-DOF model in [Equation 6].

That is, if the carbon composite material system is assumed to be an N-DOF system, then an appropriate model can be formulated using the following [Equation 7] under the similar expression shown in [Equation 6]. In addition, the normalized viscous damping coefficient in the ith mode (c̅_{eq,i}) and the normalized stiffness coefficient (k̅_{eq,i}) in the ith mode are expressed in the following [Equation 8] and [Equation 9], respectively.

$$[ \begin{matrix}1 & & zeros \\ & ⋱ & \\ zeros & & 1\end{matrix} ] \ddot{R} + [ \begin{matrix}{( \frac{1}{2 {\tilde{ς}}_{i , C} {ω}_{{n}_{i} , C}} + \frac{1}{2 {\tilde{ς}}_{i , C} {ω}_{{n}_{i} , M}} )}^{- 1} & & zeros \\ & ⋱ & \\ zeros & & {( \frac{1}{2 {\tilde{ς}}_{i , C} {ω}_{{n}_{i} , C}} + \frac{1}{2 {\tilde{ς}}_{i , C} {ω}_{{n}_{i} , M}} )}^{- 1}\end{matrix} ] \dot{R} + [ \begin{matrix}{( {ω}_{{n}_{i} , C} )}^{2} + {( {ω}_{{n}_{i} , M} )}^{2} & & zeros \\ & ⋱ & \\ zeros & & {( {ω}_{{n}_{N} , C} )}^{2} + {( {ω}_{{n}_{N} , M} )}^{2}\end{matrix} ] R = [ \begin{matrix}0 \\ ⋮ \\ 0\end{matrix} ] \qquad [ Equation 7 ]$$

$${\overline{c}}_{eq , i} = 2 {\overline{ς}}_{i , C} {ω}_{{n}_{i}} = ( {( \frac{1}{2 {\tilde{ς}}_{i , C} {ω}_{{n}_{i} , C}} + \frac{1}{2 {\tilde{ς}}_{i , C} {ω}_{{n}_{i} , M}} )}^{- 1} \qquad [ Equation 8 ]$$

$${\overline{k}}_{eq , i} = {( {ω}_{{n}_{i}} )}^{2} = {( {ω}_{{n}_{i} , C} )}^{2} + {( {ω}_{{n}_{i} , M} )}^{2} \qquad [ Equation 9 ]$$

Here, in the above [Equation 7] to [Equation 9], R=[x₁(t) x₂(t) . . . x_{N-1}(t) x_{N}(t)]^{T} is the column vector that represents the response of the carbon composite material structure. In addition, ξ_{i,C} and ξ_{i,M} are the modal damping ratio of the carbon fiber and the polymer matrix at ith mode, respectively; and ω_{ni,C} and ω_{ni,M} are the resonant frequencies of the carbon fiber and polymer matrix at ith mode, respectively.

In addition, the characteristics of the carbon composite material structure are associated closely with the carbon fiber orientation, and the system parameters shown in [Equation 8] and [Equation 9] should be addressed by considering the anisotropic mechanical characteristics.

If the carbon fibers in the carbon composite structure are assumed to be aligned at a specific angle (θⱼ), then the system parameters are functions of the carbon fiber orientation as shown in the following [Equation 10] and [Equation 11].

$${\overline{c}}_{eq , i} ( {θ}_{j} ) = {( \frac{1}{2 {\tilde{ς}}_{i , C} ( {θ}_{j} ) {ω}_{{n}_{i} , C} ( {θ}_{j} )} + \frac{1}{2 {\tilde{ς}}_{i , M} ( {θ}_{j} ) {ω}_{{n}_{i} , M} ( {θ}_{j} )} )}^{- 1} \qquad [ Equation 10 ]$$

$${\overline{k}}_{eq , i} ( {θ}_{j} ) = {( {ω}_{{n}_{i} , C} ( {θ}_{j} ) )}^{2} + {( {ω}_{{n}_{i} , M} ( {θ}_{j} ) )}^{2} \qquad [ Equation 11 ]$$

In addition, the system parameters of the above [Equation 10] and [Equation 11] can be formulated using a combination of coefficients for both the carbon fiber and the polymer matrix. According to the literature of the related art, the sensitivity of each system parameter at a certain angle θⱼ is expressed as shown in [Equation 12] and [Equation 13] below, with respect to the reference carbon fiber orientation θ₁.

$$\frac{{\overline{c}}_{C , i} ( {θ}_{j} )}{{\overline{c}}_{C , i} ( {θ}_{1} )} \approx \frac{{\overline{c}}_{eq , i} ( {θ}_{j} )}{{\overline{c}}_{eq , i} ( {θ}_{1} )} \qquad [ Equation 12 ]$$

$$\frac{{\overline{k}}_{C , i} ( {θ}_{j} )}{{\overline{k}}_{C , i} ( {θ}_{1} )} = 1 - \frac{{\overline{k}}_{eq , i} ( {θ}_{1} ) - {\overline{k}}_{eq , i} ( {θ}_{j} )}{{\overline{k}}_{eq , i} ( {θ}_{1} ) - {\overline{k}}_{eq , i} ( {θ}_{*} )} \qquad [ Equation 13 ]$$

Here, in the above [Equation 12] and [Equation 13], c̅_{C,i}(θ₁) and c̅_{C,i}(θⱼ) are the viscous damping coefficients of the carbon fiber at θ₁ and θⱼ, respectively, c̅_{eq,i}(θ₁) and c̅_{eq,i} (θⱼ) are the equivalent viscosity damping coefficient of the carbon fiber at θ₁ and θⱼ, respectively, k̅_{C,i}(θ₁) and k̅_{C,i}(θⱼ) are the stiffness coefficient of the carbon fiber at θ₁ and θⱼ, respectively, k̅_{eq,i}(θ₁) and k̅_{eq,i}(θⱼ) are the equivalent stiffness coefficient of the carbon fiber at θ₁ and θⱼ, respectively, is the lowest value of the stiffness modulus among the candidate carbon fiber orientations in the ith mode.

The equivalent system parameters can be predicted using the above [Equation 12] and [Equation 13]. If the system parameter at θ₁ and the ratio of each parameter in the left term

$$( \frac{{\overline{c}}_{C , i} ( {θ}_{j} )}{{\overline{c}}_{C , i} ( {θ}_{1} )} , \frac{{\overline{k}}_{C , i} ( {θ}_{j} )}{{\overline{k}}_{C , i} ( {θ}_{1} )} )$$

can be identified, then the equivalent modal parameters of the carbon composite structure can be predicted at a certain angle of the carbon fiber θⱼ.

Meanwhile, the viscous damping coefficient c̅_{eq,i}(θⱼ) can be predicted using the ratio of the viscous damping ratio of the carbon fiber and c̅_{eq,i}(θ₁). The prediction k̅_{C,i}(θⱼ) is not as simple as that of c̅_{eq,i}(θ₁) because it requires two equivalent stiffness coefficient, k̅_{eq,i}(θ₁) and k̅_{eq,i}(θⱼ) stiffness ratio of the carbon fiber. Therefore, the case involving the stiffness coefficient was reviewed as shown in [Equation 14] based on below [Equation 13] presented in the literature of the related art and the like.

$$\frac{{\overline{k}}_{C , i} ( {θ}_{j} )}{{\overline{k}}_{C , i} ( {θ}_{1} )} = 1 - \frac{{\overline{k}}_{eq , i} ( {θ}_{1} ) - {\overline{k}}_{eq , i} ( {θ}_{j} )}{{\overline{k}}_{C , i} ( {θ}_{1} )} \qquad [ Equation 14 ]$$

Here, the equivalent stiffness coefficient is a series combination of the stiffness coefficients of the carbon fiber and polymer matrix (see Equation 5 above), and it can be assumed that the stiffness coefficient is maximum at the reference angle θ₁, and also, as discussed in the literature of the related art, the stiffness coefficient of the polymer matrix is not affected by the carbon fiber orientation, and the stiffness coefficient of the polymer matrix is small compared with that of carbon fiber. Therefore, k̅_{C,i}(θ₁) can be replaced by k̅_{eq,i}(θ₁) under the minimum error owing to the maximum stiffness condition at θ₁, and the above [Equation 14] can be reformulated as in the following [Equation 15].

$$\frac{{\overline{k}}_{C , i} ( {θ}_{j} )}{{\overline{k}}_{C , i} ( {θ}_{1} )} \approx 1 - \frac{{\overline{k}}_{eq , i} ( {θ}_{1} ) - {\overline{k}}_{eq , i} ( {θ}_{j} )}{{\overline{k}}_{eq , i} ( {θ}_{1} )} = \frac{{\overline{k}}_{eq , i} ( {θ}_{j} )}{{\overline{k}}_{eq , i} ( {θ}_{1} )} \qquad [ Equation 15 ]$$

In addition, the increase or decrease of the equivalent system parameter are not always started from the reference angle θ₁. Therefore, another carbon fiber orientation ϕ₁ is introduced as the starting point of the variation of the system parameters. The variation of the equivalent system parameters can be efficiently identified by the rearrangement of the system parameter according to the increase of ϕⱼ, which is the jth increased carbon fiber orientation from ϕ₁.

If one system parameter is increased or decreased from the carbon fiber orientation θ₁, then the other system parameter will increase of decrease, respectively, because the relationship between the two system parameters is proportional. The ratio of the viscous damping coefficient and the stiffness coefficient in the ith mode, as shown in [Equation 12] and [Equation 15] above, respectively, can be approximately expressed by the curve-fitting function from all the carbon fiber orientation sets and estimated fitting curves were defined as [Equation 16] and [Equation 17] below, respectively.

$${Γ}_{\overline{c} , i} ( {ϕ}_{j} ) \approx \frac{{\overline{c}}_{C , i} ( {ϕ}_{j} )}{{\overline{c}}_{C , i} ( {ϕ}_{1} )} \qquad [ Equation 16 ]$$

$${Γ}_{\overline{k} , i} ( {ϕ}_{j} ) \approx \frac{{\overline{k}}_{C , i} ( {ϕ}_{j} )}{{\overline{k}}_{C , i} ( {ϕ}_{1} )} \qquad [ Equation 17 ]$$

As shown in [Equation 12] and [Equation 15], the equivalent system parameters, viscous damping coefficient, and stiffness coefficient of the carbon composite material structure can be directly predicted from the curve-fitting functions Γ_{c̅,i} and Γ_{k̅,i}. However, the main focus of the present disclosure is to estimate the system parameters at certain carbon fiber orientations using the measured data at a reference carbon fiber only without requiring any further measurement data sets.

Here, one of the methods for predicting system parameters is to introduce a representative curve-fitting function if the representative function can encompass all modes of interest in the carbon composite material structure. Assuming that two representative functions defined by Γ_{c̅} and Γ_{k̅} can be applied to the prediction of system parameters, viscous damping coefficients, and stiffness coefficients, the approximated system parameters in a certain carbon fiber orientation (ϕⱼ) can be expressed as shown in [Equation 18] and [Equation 19].

c̅_{eq,i}(ϕⱼ)=c̅_{eq,i}(ϕ₁)Γ_{c̅,i}(ϕⱼ)≈c̅_{eq,i}(ϕ₁)Γ_{c̅}(ϕⱼ) [Equation 18]

k̅_{eq,i}(ϕⱼ)=k̅_{eq,i}(ϕ₁)Γ_{k̅,i}(ϕᵢ)≈k̅_{eq,i}(ϕ₁)Γ_{k̅}(ϕⱼ) [Equation 19]

As described above, according to the present disclosure, normalized viscous damping coefficient and structural stiffness coefficient are respectively obtained by dividing by the modal mass at the ith mode with respect to the carbon angle (θⱼ) of the carbon composite material as described above with reference to [Equation 10] and [Equation 11]. As described above with reference to [Equation 12] to [Equation 15], the relationship between the damping coefficient and the structural stiffness for the reference carbon fiber angle (θ₁) and the arbitrary angle (θⱼ) is shown and introduces a new carbon fiber angle variable ϕⱼ as described above with reference to [Equation 16] and [Equation 17], and the increase or decrease of the system parameters (damping coefficient, stiffness coefficient) based on ϕ₁ is approximated through curve-fitting function. From approximations for damping coefficients and stiffness coefficients, a series of processes may be performed to predict system parameters for any carbon fiber orientation as described above with reference to [Equations 18] and [Equations 19]. In this case, the necessary information is sufficient only with the approximation equation for each variable and the parameter information for the reference angle ϕ₁.

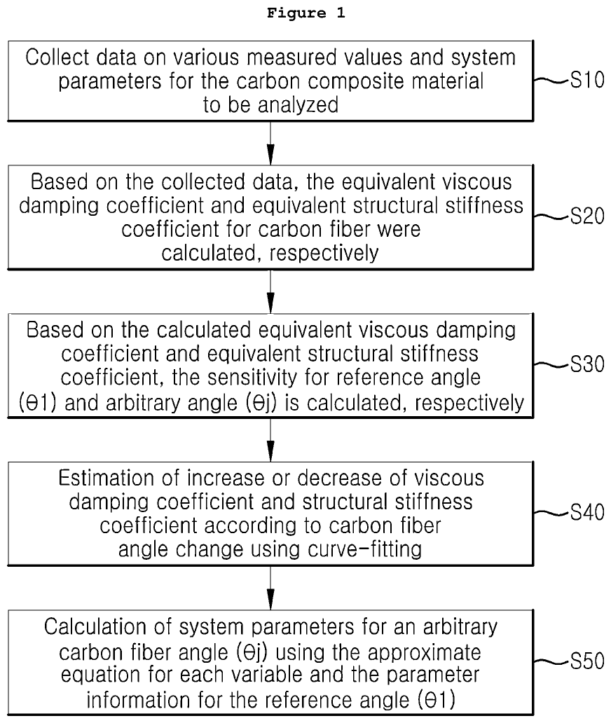

FIG. 1 is a flowchart schematically showing the overall configuration of a method for analyzing the dynamic characteristics of a carbon composite material according to an angle of carbon fiber according to an embodiment of the present disclosure.

As shown in FIG. 1, the method for analyzing the dynamic characteristics of the carbon composite material according to the carbon fiber angle according to the embodiment of the present disclosure includes largely dividing: a data collection step (S10) in which a process of collecting data on various predetermined measurement values and system parameters is performed on a carbon composite material to be analyzed; a system parameter calculation step (S20) in which a process of calculating a viscosity damping coefficient and a rigidity coefficient for a carbon angle of the carbon composite material to be analyzed is performed based on the data collected in the data collection step (S10); a sensitivity calculation step (S30) in which a process of calculating the sensitivity of the viscosity damping coefficient and the structural stiffness coefficient is performed based on the viscosity damping coefficient and the structural stiffness coefficient calculated in the system parameter calculation step (S20); a system parameter approximation step (S40) in which a process of approximating a change in viscosity damping coefficient and structural stiffness coefficient according to a change in a carbon fiber angle is performed using a curve-fitting method based on the sensitivity calculated in the sensitivity calculation step (S30); a system parameter estimation step (S50) in which a process for estimating a system parameter for an arbitrary carbon fiber orientation is performed based on the processing result of the system parameter approximation step (S40). A series of processes, including the steps described above, may be configured to be performed through a computer or dedicated hardware.

Here, the data collection step is configured to perform a modal test to collect various predetermined system parameters or to receive data collected in advance through the modal test after preparing a specimen having a preset carbon fiber angle (θ₁) with respect to the carbon composite material to be analyzed.

In addition, the system parameter calculation step S20 may be configured to perform a process of calculating an equivalent viscosity damping coefficient and an equivalent stiffness coefficient in the ith mode, respectively, using [Equation 10] and [Equation 11].

In addition, the sensitivity calculation step (S30) may be configured to perform a process of calculating the sensitivity of the viscosity damping coefficient and the structural stiffness coefficient at a specific angle θⱼ for the reference carbon fiber angle θ₁ using the [Equation 12] and [Equation 15].

Furthermore, the system parameter approximation step S40 may be configured to perform the process of approximating each and rearranging each system parameter using the [Equation 16] and [Equation 17], in which the increase or decrease of the viscous damping coefficient and the structural stiffness coefficient at the jth increased carbon fiber angle ϕⱼ from the reference carbon fiber angle ϕ₁ using a curve-fitting function.

In addition, the system parameter estimation step (S50) may be configured to perform a process of estimating system parameters for an arbitrary carbon fiber orientation using the above [Equation 18] and [Equation 19], thereby, a system parameter for an arbitrary carbon fiber orientation may be estimated only with the parameter information on the approximation function for each variable and the reference angle ϕ₁.

Subsequently, the dynamic characteristics analysis method of the carbon composite material according to the carbon fiber angle according to the embodiment of this disclosure configured as described above will be described through experiments.

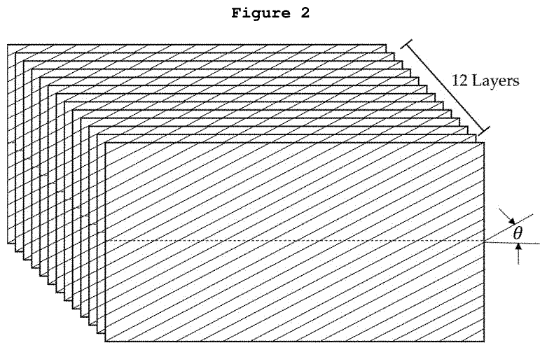

First, referring to FIG. 2, FIG. 2 is a view schematically showing the overall configuration of the carbon composite material specimen produced for the verification test of the method for analyzing the dynamic characteristics of the carbon composite material according to the carbon fiber angle according to an embodiment of the present disclosure.

As shown in FIG. 2, the present inventors, in order to verify the dynamic characteristic analysis method of the carbon composite material according to the carbon fiber angle according to the embodiment of the present disclosure as described above, a specimen of a unidirectional rectangular shape (80 mm(W)×150 mm(L)×3 mm(H)) was manufactured using 12 layers of the pre-impregnated material (USN 250A, SK Chemicals, Seongnam, Korea) and the modal test was conducted, and changes in system parameters were respectively shown based on θ₁, which is the reference carbon fiber orientation, for five modes (three bending modes) and two torsional modes, respectively.

Here, USN 250A is composed of T700 carbon fiber (12k, Toray, Tokyo, Japan) and a polymer matrix (epoxy resin) and cured under an autoclave process (up to 125° C.), and five specimens were prepared, and a modal test was performed by varying the carbon fiber angles of θ₁=0°, θ₂=30°, θ₃=45°, θ₄=60°, and θ₅=90°.

In addition, the modal parameters of each specimen were measured using an experimental impact test technique, an impact hammer (model: 5800B3, Dytran, Chatsworth, Calif., USA) and uniaxial accelerometers (model: 3225F2), Dytran) was used to measure the frequency response function (FRF) between the impact point and the response locations.

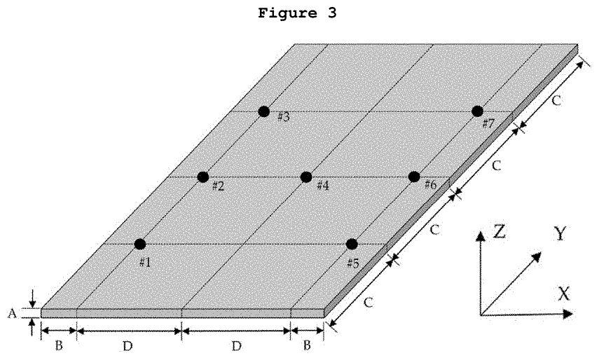

More specifically, referring to FIG. 3, FIG. 3 is a view schematically showing a position of a sensor attached to a specimen.

In FIG. 3, A is 3 mm, B is 10 mm, C is 37.5 mm, D is 30 mm, an impact force was applied to #4, and response acceleration was measured at positions #1 to #7, respectively.

Here, the total mass of the specimen was 56.5 (g), but the total mass of the accelerometer was 1 (g)×7=7 (g), so the mass loading effect of the accelerometer was negligible, and the locations of the sensor attached to the specimen were chosen to leave sufficient space between the respective sensors.

In addition, the frequency range was selected between 10 Hz and 4096 Hz, and 10 times the average of the measured FRF was used to remove noise from the FRF of the specimen. The constraint condition of the specimen was set to free-free using a rubber band, and all data was recorded using an 8-channel data acquisition device (Test. Lab/Siemens/Germany).

In addition, the system parameters of the specimen were calculated using the PolyMax algorithm of Test. Lab equipment. At this time, since the resonance frequency and the corresponding eigenvector change depending on the direction of the carbon fiber, the change in the resonance frequency and viscous damping coefficient suggested in previous studies, etc., as well as the mode of interest was tracked using the Modal Assurance Criterion (MAC).

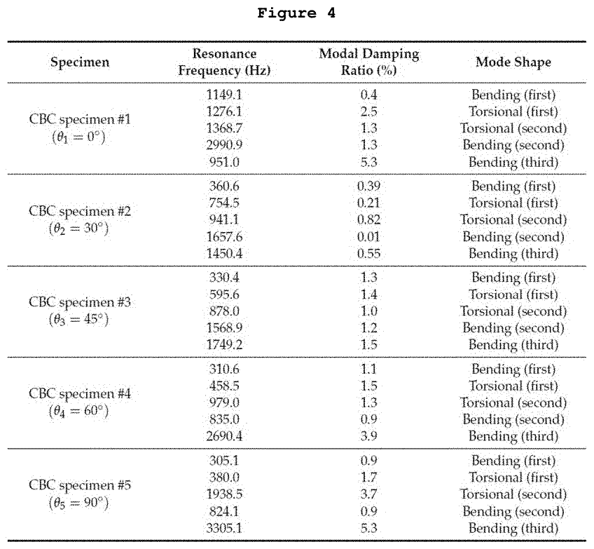

Here, the MAC value was calculated using MATLAB software (MathWorks, Natick, Mass., USA), and the tracked five modes of interest and changes in each system parameter are shown in FIGS. 4 to 6B, respectively.

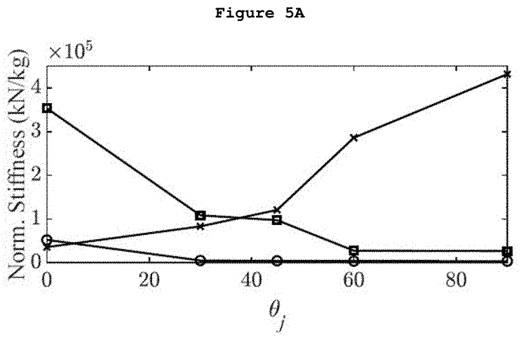

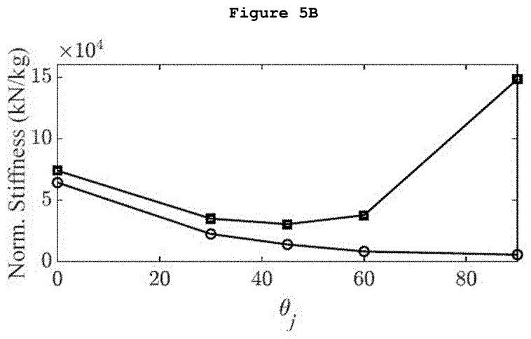

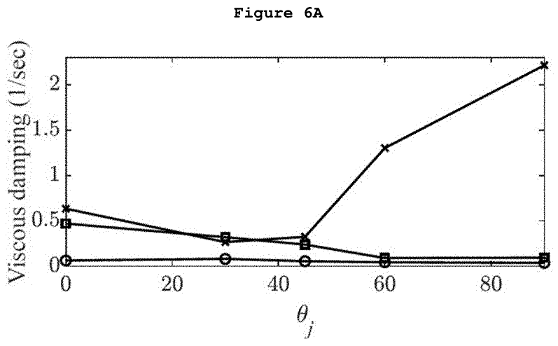

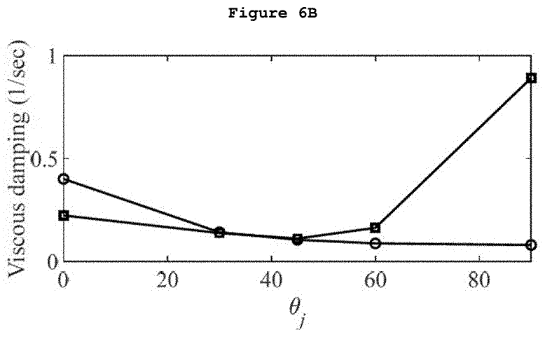

That is, FIG. 4 is a view showing the mode parameters measured for each specimen in five carbon fiber orientations in a table, and FIGS. 5A, 5B, 6A, and 6B are graphs showing a change in structural stiffness and a change in viscosity damping coefficient according to the direction of carbon fiber, respectively.

FIGS. 5A and 6A each represent a bending mode, and [symbol] is a primary bending mode, [symbol] is a secondary bending mode, [symbol] is a tertiary bending mode, respectively, FIGS. 5B and 6B, respectively, denote a torsional mode, [symbol] is a primary torsional mode and [symbol] is a secondary torsional mode, respectively.

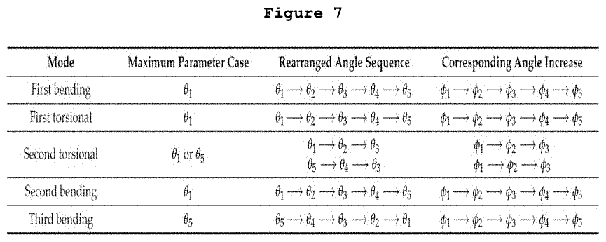

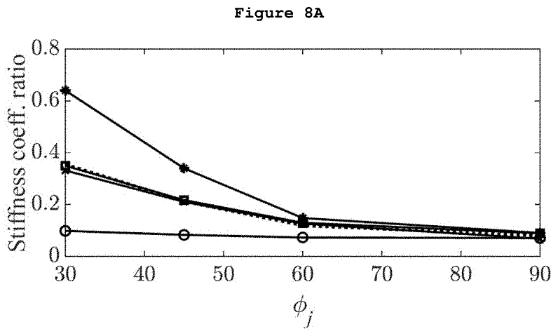

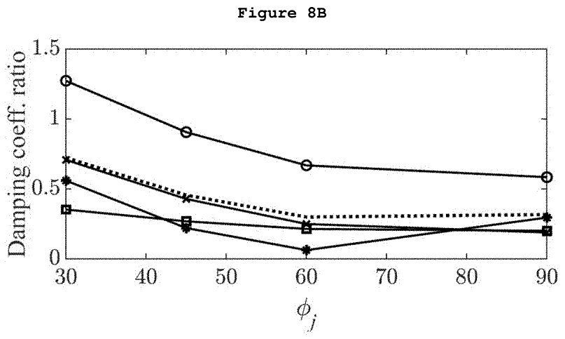

In addition, FIG. 7 shows the increase or decrease of the structural stiffness and the viscous damping coefficient shown in the graphs of FIGS. 5A, 5B, 6A and 6B using the carbon fiber angle variable θⱼ proposed in the present disclosure. FIGS. 8A and 8B are graphs showing changes in structural stiffness and viscous damping coefficient by using the carbon fiber angle variable θⱼ by rearranging them through a curve-fitting method.

Here, FIG. 8A shows the change in stiffness coefficient ratio, [symbol] is the primary bending mode, [symbol] is the primary torsional mode, [symbol] is the secondary bending mode, [symbol] is the tertiary bending mode, [symbol] is the averaged curve, respectively, and FIG. 8B shows the change in damping coefficient ratio, [symbol] is the primary bending mode, [symbol] is the primary torsional mode, [symbol] is the secondary bending mode, [symbol] is the tertiary bending mode, [symbol] is the averaged curve.

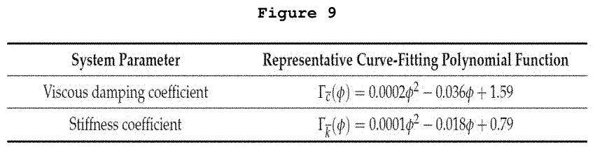

FIG. 9 is a table showing representative curve-fitting functions for each system variable obtained using a curve-fitting function.

Here, in FIG. 9, a representative approximation equation is shown by averaging the coefficients of each approximation equation for five modes.

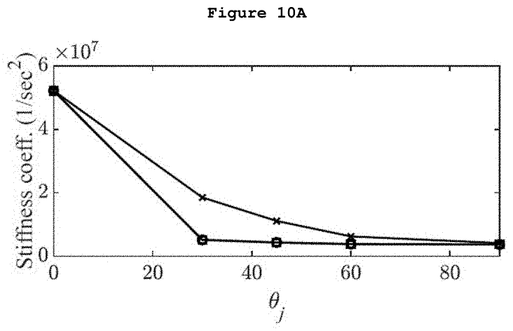

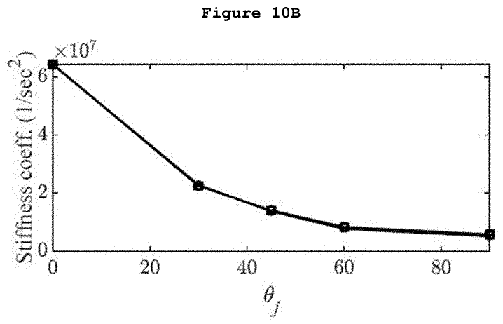

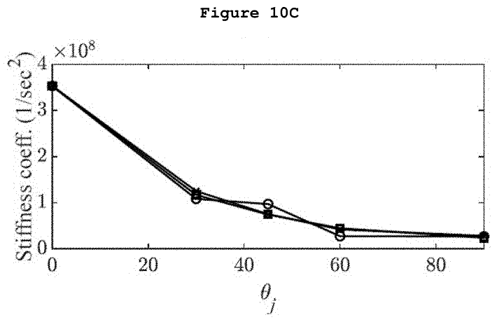

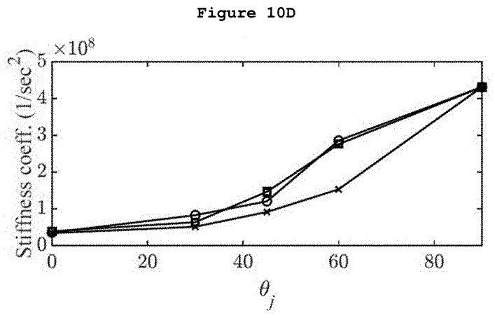

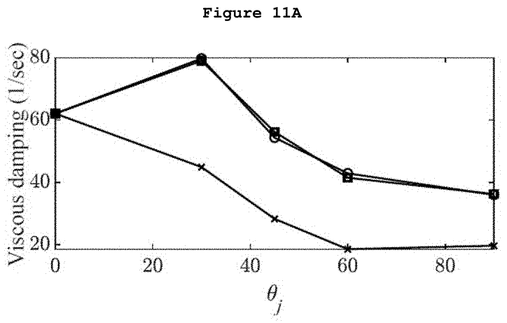

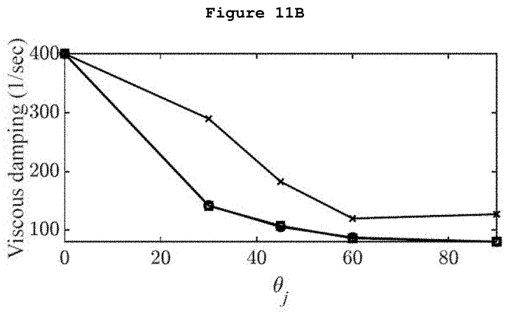

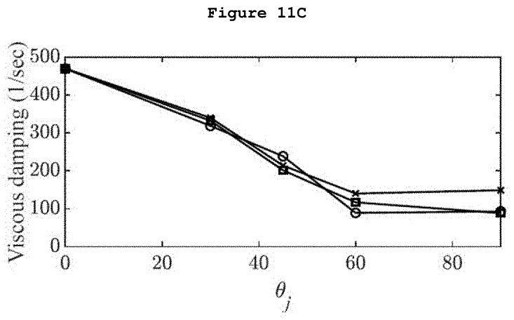

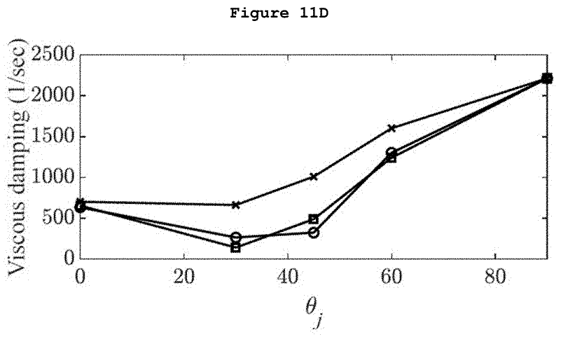

Subsequently, FIGS. 10A-10D and 11A-11D are diagrams showing the results of comparing the predicted parameter change according to the carbon fiber angle predicted using the representative approximation equation shown in FIG. 9 with the actual measured value. FIGS. 10A-10D are comparison results for the stiffness coefficient, and FIGS. 11A-11D show a comparison result for the viscous damping coefficient, respectively.

Here, in FIGS. 10A-10D, [symbol] is a measured parameter, [symbol] is a parameter predicted using a curve-fitting function, [symbol] is a parameter predicted through a representative approximation equation, respectively, and FIG. 10A is a first mode (primary bending), FIG. 10B shows the second mode (primary torsional), FIG. 10C shows the fourth mode (secondary bending) and FIG. 10D shows the fifth mode (tertiary bending), respectively.

In addition, in FIGS. 11A-11D, [symbol] is a measured parameter, [symbol] is a parameter predicted using a curve-fitting function, [symbol] is a parameter predicted through a representative approximation equation, respectively, FIG. 11A is a first mode (primary bending), FIG. 11B shows a second mode (primary torsional), FIG. 11C shows a fourth mode (secondary bending), and FIG. 11D shows a fifth mode (tertiary bending), respectively.

As shown in FIGS. 10A-10D and 11A-11D, the present inventors compared changes in system parameters according to the predicted carbon fiber angle with actual measured values using a representative approximation equation, and obtained approximate equations for each of the four modes and compared them, and the result is direct prediction. The results using the representative approximation equations for stiffness and damping shown in the table of FIG. 9 were named as indirect predictions, respectively.

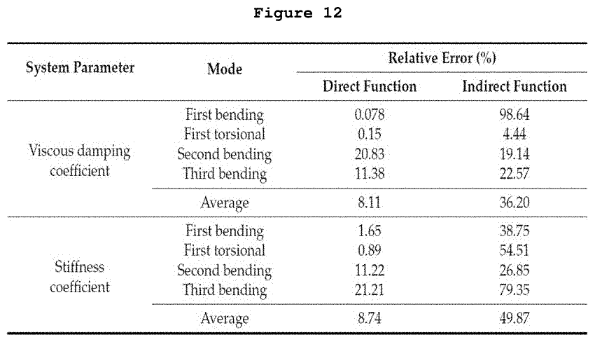

FIG. 12 is a view showing the results of calculating a relative error with respect to the actual measurement data in order to compare the accuracy of the actual measurement data and the predicted data in a table.

As shown in FIG. 12, after calculating the approximate equations for each of the four modes and comparing them with the measured data, the average error was 8.11% (viscous damping coefficient) and 8.74% (stiffness coefficient), respectively, it may be confirmed indicating relatively high reliability.

Here, the result using the representative approximation equation proposed in the present disclosure shows a rather high error of 36.2% (viscous damping coefficient) and 49.87% (stiffness coefficient), but this is an expected condition using data only from the reference angle of carbon fiber. Therefore, the result using the representative approximation equation can provide a sufficiently useful result to be used as a design guideline.

That is, the direct prediction method for each mode can provide very accurate information, but it requires a lot of detailed data, so in some cases, using the test data as it is may be more economical.

Meanwhile, the indirect prediction method has a relatively large error compared to the direct prediction, but the prediction result is equally applicable to all modes, so the indirect prediction method has sufficient engineering value. Therefore, the present disclosure may be said to be an efficient method for predicting dynamic characteristics of carbon composite materials under other carbon fiber angle conditions by using the limited information such as system parameter information at a reference angle.

Here, in the above-described embodiment of the present disclosure, for more detailed information about the process of measuring and calculating data for various system parameters of the carbon composite material through a modal test, for example, it is a matter that may be appropriately configured by those skilled in the art with reference to the contents as presented in “Method for calculating sensitivity index for structural stiffness and viscous damping coefficient of carbon composite material and method for analysis of dynamic characteristics of carbon composite material using the calculation method” of Korean Patent Application No. 10-2021-0143860, filed as 2021.10.26, and the modal test apparatus or method of the related art. Therefore, in the present disclosure, in order to simplify the description, it should be noted that the detailed description thereof that may be easily understood and performed by those skilled in the art is omitted with reference to the literature of the prior art such as the modal test.

In addition, according to the present disclosure, it is possible to easily implement a system for analyzing the dynamic characteristics of a carbon composite material by using the method for analyzing the dynamic characteristics of the carbon composite material according to the carbon fiber angle according to the embodiment of the present disclosure configured as described above.

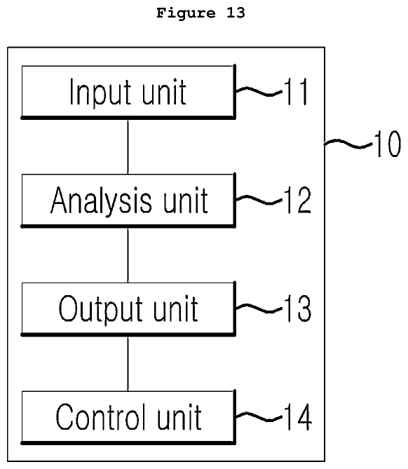

More specifically, referring FIG. 13, FIG. 13 is a block diagram schematically showing the overall configuration of the carbon composite material specimen produced for the verification test of the method for analyzing the dynamic characteristics of the carbon composite material according to the carbon fiber angle according to an embodiment of the present disclosure.

As shown in FIG. 13, the dynamic characteristic analysis system 10 of the carbon composite material according to the embodiment of the present disclosure is largely divided, including: an input unit 11 for receiving various data including the modal test result of the carbon composite material to be analyzed and information on each system parameter; an analysis unit 12 in which a process of estimating a system parameter according to a change in the angle of carbon fiber of a carbon composite material to be analyzed is performed based on data input through the input unit 11; an output unit 13 for outputting data input through the input unit 11 and a processing result of the analysis unit 12; and a control unit 14 for controlling the overall operation of each of the above-described parts and systems.

Here, the above-described analysis unit 12, using the dynamic characteristic analysis method of the carbon composite material according to the carbon fiber angle according to the embodiment of the present disclosure described above with reference to FIGS. 1 to 12 may be configured to perform a process of estimating a system parameter at an arbitrary specific carbon fiber angle θⱼ only with parameter information about the reference carbon fiber angle, θ₁.

Moreover, the dynamic characteristic analysis system 10 of the carbon composite material described above may be configured by installing a dedicated program in an information processing device such as a PC or a notebook computer, and thus may be implemented in a simpler configuration and at lower costs without requiring separate hardware.

That is, the method for analyzing the dynamic characteristics of the carbon composite material according to the carbon fiber angle according to the embodiment of the present disclosure is a computer program configured to execute a series of processing steps as described above with reference to FIGS. 1 to 12 recorded on the computer. It should be noted that this disclosure may be configured in various forms as necessary, such as providing such computer programs in the form of computer-readable recording media on which they are recorded.

As described above, a method for analyzing dynamic characteristics of a carbon composite material according to an angle of a carbon fiber according to an embodiment of the present disclosure may be implemented. According to the present disclosure, with respect to the structural stiffness and viscous damping coefficient, which are system parameters very sensitive to the carbon fiber orientation among the dynamic characteristics of the carbon composite material, values of structural stiffness and viscosity damping coefficient for carbon fiber orientation of other angles can be predicted, respectively, using only the data of the carbon fiber orientation for the reference angle. Accordingly, provided is a method for analyzing dynamic characteristics of a carbon composite material according to an angle of a carbon fiber configured to predict system parameters of carbon composite materials for various carbon fiber orientations and reflect them in the design of carbon composite materials using only single carbon fiber orientation data. Therefore, before making a product, various system variables may be predicted in advance in the design step of the carbon composite material to determine the carbon fiber orientation having desired dynamic characteristics easily.

In addition, according to the present disclosure, provided is a method for analyzing dynamic characteristics of a carbon composite material according to an angle of a carbon fiber configured to accurately predict system parameters of a carbon composite material having various angles in a relatively simple configuration using only data in a single carbon fiber orientation, as described above. A method for determining the direction of carbon fiber having desired dynamic characteristics by predicting various system variables in advance in the design stage of carbon composite material before making a product, in which most of the process is to produce specimens for specific carbon fiber orientations and perform characteristic tests to obtain and analyze data, has not been presented and can solve the limitations of the methods for analyzing the dynamic characteristics of carbon composite materials of the related art.

In the embodiments of this disclosure as described above, the method for analyzing the dynamic characteristics of carbon composite materials according to the angle of carbon fiber in accordance with this disclosure has been described. However, the present disclosure is not limited to the contents described in the above embodiment, and thus it is natural that the present disclosure may be modified, modified, combined, and replaced according to design needs and various other factors by a person skilled in the art.

What is claimed is:

1. A method of analyzing dynamic characteristics of carbon composite materials according to orientations of carbon fiber, the method being performed by a computer or a dedicated hardware unit, the method comprising:
collecting data on various predetermined measurement values and system parameters for a carbon composite material to be analyzed;
deriving a viscous damping coefficient and a stiffness coefficient for a carbon angle of the carbon composite material to be analyzed, from the collected data;
calculating a sensitivity level of the viscosity damping coefficient and a sensitivity level of the structural stiffness coefficient on the basis of the derived viscosity damping coefficient and the derived structural stiffness coefficient;
approximating changes in the viscous damping coefficient and the structural stiffness coefficient according to a change in the carbon fiber orientation using a curve-fitting method on the basis of the calculated sensitivity levels; and
estimating a system parameter for an arbitrary carbon fiber orientation on the basis of the result of the system parameter approximation.

2. The method of claim 1, wherein the collecting comprises:
preparing a specimen having a predetermined reference carbon fiber angle (θ₁) for the carbon composite material to be analyzed and collecting various predetermined system parameters by performing a modal test on the specimen; or
receiving data that are collected through a previous model test.

3. The method of claim 2, wherein the deriving comprises:
calculating each of a normalized equivalent viscosity attenuation coefficient (C̅_{eq,i}) and an equivalent stiffness coefficient (k̅_{eq,i}) for the ith mode, using the following equation,
$${\overline{c}}_{eq , i} ( {θ}_{j} ) = {( \frac{1}{2 {\tilde{ς}}_{i , C} ( {θ}_{j} ) {ω}_{{n}_{i} , C} ( {θ}_{j} )} + \frac{1}{2 {\tilde{ς}}_{i , M} ( {θ}_{j} ) {ω}_{{n}_{i} , M} ( {θ}_{j} )} )}^{- 1}$$
$${\overline{k}}_{eq , i} ( {θ}_{j} ) = {( {ω}_{{n}_{i} , C} ( {θ}_{j} ) )}^{2} + {( {ω}_{{n}_{i} , M} ( {θ}_{j} ) )}^{2}$$
(where, θⱼ is the carbon fiber angle, ξ_{i,C} is a modal damping ratio in the ith mode, ξ_{i,M} a modal damping ratio of a polymer matrix in the ith mode, ω_{ni,C} is a resonance frequency of the carbon fiber in the ith mode, and ω_{ni,M} is a resonance frequency of the polymer matrix in the ith mode.

4. The method of claim 3, wherein the calculating comprises:
calculating the sensitivity level of the viscous damping coefficient and the sensitivity level of the structural stiffness coefficient at a specific carbon fiber angle θⱼ with respect to the reference carbon fiber angle θ₁, using the following equation
$$\frac{{\overline{c}}_{C , i} ( {θ}_{j} )}{{\overline{c}}_{C , i} ( {θ}_{1} )} \approx \frac{{\overline{c}}_{eq , i} ( {θ}_{j} )}{{\overline{c}}_{eq , i} ( {θ}_{1} )}$$
$$\frac{{\overline{k}}_{C , i} ( {θ}_{j} )}{{\overline{k}}_{C , i} ( {θ}_{1} )} = \frac{{\overline{k}}_{eq , i} ( {θ}_{j} )}{{\overline{k}}_{eq , i} ( {θ}_{1} )} ,$$
(where, C̅_{Ci}(θ₁) and C̅_{C,i}(θⱼ) are the viscous damping coefficients of the carbon fiber at θ₁ and θⱼ, respectively, C̅_{eq,i}(θ₁) and C̅_{eq,i}(θⱼ) are the equivalent viscous damping coefficients of the carbon fiber at θ₁ and θⱼ, respectively, k̅_{C,i}(θ₁) and k̅_{C,i}(θⱼ) are the stiffness modulus of the carbon fiber at θ₁ and θⱼ, respectively, k̅_{eq,i}(θⱼ) and are the equivalent stiffness modulus of carbon fiber at θ1 and θⱼ, respectively)

5. The method of claim 4, wherein the approximating comprises:
approximating an increase or decrease in each of the viscous damping coefficient and the structural stiffness coefficient at an jth increased carbon fiber angle ϕⱼ from the reference carbon fiber angle ϕ₁ using a curve-fitting function (Γ_{c̅,i}, Γ_{k̅,i}).
$${Γ}_{\overline{c} , i} ( {ϕ}_{j} ) \approx \frac{{\overline{c}}_{C , i} ( {ϕ}_{j} )}{{\overline{c}}_{C , i} ( {ϕ}_{1} )}$$
$${Γ}_{\overline{k} , i} ( {ϕ}_{j} ) \approx \frac{{\overline{k}}_{C , i} ( {ϕ}_{j} )}{{\overline{k}}_{C , i} ( {ϕ}_{1} )}$$

6. The method of claim 5, wherein the estimating comprises:
estimating system parameters for an arbitrary carbon fiber orientation using the following equation,
C̅_{eq,i}(ϕⱼ)=C̅_{eq,i}(ϕ₁)Γ_{c̅,i}(ϕⱼ)≈c_{eq,i}(ϕ₁)Γ_{c̅}(ϕⱼ)
k̅_{eq,i}(ϕⱼ)=k̅_{eq,i}(ϕ₁)Γ_{k̅,i}(ϕⱼ)≈k̅_{eq,i}(ϕ₁)Γ_{k̅}(ϕⱼ)

7. A non-transitory computer-readable recording medium storing instructions, when executed by a computer or a dedicated hardware unit, to perform a method of analyzing dynamic characteristics of carbon composite materials according to orientations of carbon fiber, the method comprising:
collecting data on various predetermined measurement values and system parameters for a carbon composite material to be analyzed;
deriving a viscous damping coefficient and a stiffness coefficient for a carbon angle of the carbon composite material to be analyzed, from the collected data;
calculating a sensitivity level of the viscosity damping coefficient and a sensitivity level of the structural stiffness coefficient on the basis of the derived viscosity damping coefficient and the derived structural stiffness coefficient;
approximating changes in the viscous damping coefficient and the structural stiffness coefficient according to a change in the carbon fiber orientation using a curve-fitting method on the basis of the calculated sensitivity levels; and
estimating a system parameter for an arbitrary carbon fiber orientation on the basis of the result of the system parameter approximation.

8. A system for analyzing dynamic characteristics of carbon composite materials, the system comprising:
an input unit configured to receive various kinds of data including information on each system parameter of a carbon composite material to be analyzed, and a modal test result;
an analysis unit configured to estimate a system parameter according to a change in the carbon fiber angle of the carbon composite material to be analyzed on the basis of the data input through the input unit;
an output unit configured to output the that is data input through the input unit and to output a processing result of the analysis unit; and
a controller configured to control the overall operation of the analysis system,
wherein the analysis unit is configured to estimate system parameters according to changes in the carbon fiber angle of the carbon composite material to be analyzed by using the method of claim 1.

9. The system of claim 8, wherein the system is configured to be constructed by installing a dedicated program in an information processing device including a personal computer or a laptop computer.
